SEQUENCE LISTING (13 sequences)

SEQ ID NO: 1 - protein, 124 aa, synthetic construct
EVQLVESGGGLVQPGGSLRLSCAASGFTFDDYAMSWVRQAPGKGLEWIGRMYNNGRTSYNPSLKSLVTISRDNSKNTLYLQMNSLRAEDTATYYCARDNLGYRPSENLYGMDVWGQGTTVTVSS

SEQ ID NO: 2 - DNA, 372 bp, synthetic construct
  source 1..372: mol_type = other DNA
gaagtacaacttgttgaatccggtggcggattagtacagccaggcggatctttgagattgtcctgtgctgctagcggattcacttttgacgattatgctatgagttgggttagacaagctcctggcaagggtctcgagtggatcggcagaatgtataacaatggccgtacatcttacaatccttctctcaaaagtttggttactatcagcagagataattccaaaaacaccctttacttgcagatgaactcacttagggcagaggacactgctacttattactgcgcaagagataaccttggttacaggccatctgaaaatctttatggcatggatgtttggggccaaggtactaccgtgacagtatcttca

SEQ ID NO: 3 - protein, 237 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGGGSGGHHHHHH

SEQ ID NO: 4 - protein, 154 aa, synthetic construct
EVQLVESGGGLVQPGGSLRLSCAASGFTFDDYAMSWVRQAPGKGLEWIGRMYNNGRTSYNPSLKSLVTISRDNSKNTLYLQMNSLRAEDTATYYCARDNLGYRPSENLYGMDVWGQGTTVTVSSAAAEQKLISEEDLNSAVDHHHHHHHHHHHH

SEQ ID NO: 5 - protein, 146 aa, synthetic construct
EVQLVESGGGLVQPGGSLRLSCAASGFTFDDYAMSWVRQAPGKGLEWIGRMYNNGRTSYNPSLKSLVTISRDNSKNTLYLQMNSLRAEDTATYYCARDNLGYRPSENLYGMDVWGQGTTVTVSSEQKLISEEDLHHHHHHHHHHHH

SEQ ID NO: 6 - protein, 154 aa, synthetic construct
EVQLVESGGGLVQPGGSLRLSCAASGFTFDDYAMSWVRQAPGKGLEWIGRMYNNGRTSYNPSLKSLVTISRDNSKNTLYLQMNSLRAEDTATYYCARDNLGYRPSENLYGMDVWGQGTTVTVSSAAAEQKLISEEDLNSVADHHHHHHHHHHHH

SEQ ID NO: 7 - DNA, 25 bp, synthetic construct
  source 1..25: mol_type = other DNA
gaagtacaacttgttgaatccggtg

SEQ ID NO: 8 - DNA, 25 bp, synthetic construct
  source 1..25: mol_type = other DNA
tgaagatactgtcacggtagtacct

SEQ ID NO: 9 - protein, 231 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGGGSGG

SEQ ID NO: 10 - protein, 142 aa, synthetic construct
EVQLVESGGGLVQPGGSLRLSCAASGFTFDDYAMSWVRQAPGKGLEWIGRMYNNGRTSYNPSLKSLVTISRDNSKNTLYLQMNSLRAEDTATYYCARDNLGYRPSENLYGMDVWGQGTTVTVSSAAAEQKLISEEDLNSAVD

SEQ ID NO: 11 - protein, 134 aa, synthetic construct
EVQLVESGGGLVQPGGSLRLSCAASGFTFDDYAMSWVRQAPGKGLEWIGRMYNNGRTSYNPSLKSLVTISRDNSKNTLYLQMNSLRAEDTATYYCARDNLGYRPSENLYGMDVWGQGTTVTVSSEQKLISEEDL

SEQ ID NO: 12 - protein, 142 aa, synthetic construct
EVQLVESGGGLVQPGGSLRLSCAASGFTFDDYAMSWVRQAPGKGLEWIGRMYNNGRTSYNPSLKSLVTISRDNSKNTLYLQMNSLRAEDTATYYCARDNLGYRPSENLYGMDVWGQGTTVTVSSAAAEQKLISEEDLNSVAD

SEQ ID NO: 13 - DNA, 372 bp, synthetic construct; identical to SEQ ID NO: 2
  source 1..372: mol_type = other DNA

(19) United States
(12) Patent Application Publication
(10) Pub. No.: US 2024/0401070 A1
(43) Pub. Date: Dec. 5, 2024

(54) PRODUCTION OF SINGLE DOMAIN COVID-19 ANTIBODIES IN PLANT CELLS

(71) Applicant: Rutgers, The State University of New Jersey, New Brunswick, NJ (US)

(72) Inventors: Tamara Minko, East Brunswick, NJ (US); Natalia Pogrebnyak, Monroe Township, NJ (US); Andrew M. Shen, East Brunswick, NJ (US); Obeid M. Malekshah, Somerset, NJ (US)

(73) Assignee: Rutgers, The State University of New Jersey, New Brunswick, NJ (US)

(21) Appl. No.: 18/680,246

(22) Filed: May 31, 2024

Related U.S. Application Data
(60) Provisional application No. 63/505,204, filed on May 31, 2023.

(51) Int. Cl. C12N 15/82; C07K 16/10; C12N 5/04; C12N 15/74

(52) U.S. Cl. CPC C12N 15/8258; C07K 16/1003; C12N 5/04; C12N 15/743; C07K 2317/569

(57) ABSTRACT

Methods for producing single N-terminal domain functional camelid-derived heavy (H)-chain antibody fragments (VHH) that specifically bind to a receptor binding domain (RBD) of SARS-CoV-2 spike protein include transgenic plants, plant parts, plant cells, plant tissue, and seeds that express the VHH that specifically binds to an RBD of SARS-CoV-2 spike protein.

### CROSS-REFERENCE TO RELATED APPLICATION

The present application claims the benefit of priority under 35 U.S.C. § 119(e) of U.S. Provisional Application Ser. No. 63/505,204, filed May 31, 2023. The entire disclosure of which is incorporated herein by reference.

### FIELD OF THE INVENTION

The invention relates generally to the fields of medicine, molecular biology, and transgenic plants. In particular, the invention relates to methods and compositions for producing single domain COVID-19 antibodies in plant cells.

### REFERENCE TO AN ELECTRONIC SEQUENCE LISTING

The contents of the electronic sequence listing (SeqList-07043901829.xml; Size: 14,471 bytes; and Date of Creation: May 30, 2024) is herein incorporated by reference in its entirety.

### BACKGROUND OF THE INVENTION

Data show a decrease in the risk of hospitalization and death from COVID-19 when an individual is vaccinated against SARS-CoV-2. To date, global vaccinations for SARS-CoV-2 protection are underway, but additional treatments are urgently needed to prevent and cure infection among naïve and even vaccinated people. Neutralizing monoclonal antibodies are very promising for prophylaxis and therapy of SARS-CoV-2 infections. However, traditional large-scale methods of producing such antibodies are slow, extremely expensive and possess a high risk of contamination with viruses, prions, oncogenic DNA and other pollutants.

### SUMMARY

Described herein are methods for producing VHH that specifically binds to a receptor binding domain (RBD) of SARS-CoV-2 spike protein. Also described herein are transgenic plants, plant parts (e.g., leaves), plant cells, plant tissue, and seeds that express the VHH that specifically binds to a RBD of SARS-CoV-2 spike protein. The presently claimed invention is a novel approach of producing monoclonal antibodies (mAbs) against SARS-CoV-2 spike (S) protein in plant systems which offers unique advantages, such as the lack of human and animal pathogens or bacterial toxins, relatively low-cost manufacturing, and ease of production scale-up. In the experiments described herein, a single N-terminal domain functional camelid-derived heavy (H)-chain antibody fragments (VHH, also referred to as “nanobodies”) targeted to the receptor binding domain of SARS-CoV-2 spike protein was selected and methods of its rapid production using transgenic plants and plant cell suspensions were developed. Isolated and purified plant-derived VHH antibodies were compared with mAbs produced in traditional mammalian and bacterial expression systems. It was found that plant-generated VHH using the proposed methods of transformation and purification possess the ability to bind to SARS-CoV-2 spike protein comparable to that of monoclonal antibodies derived from bacterial and mammalian cell cultures. The results of the experiments described herein confirm the viability of producing monoclonal single-chain antibodies with a high ability to bind the targeted COVID-19 spike protein in plant systems within a relatively shorter time span and at a lower cost when compared with traditional methods. Moreover, these methods can be used for producing monoclonal neutralizing antibodies against other types of viruses.

Accordingly, described herein is a transgenic plant or plant tissue or plant cell, or progeny thereof, that includes at least one polynucleotide integrated into the nuclear genome of the plant or plant tissue or plant cell, or progeny thereof, the at least one polynucleotide including at least one nucleotide sequence encoding a single variable domain of a heavy-chain antibody fragment (VHH) that specifically binds to a receptor binding domain of SARS-CoV-2 spike protein. The at least one polynucleotide includes a nucleotide sequence encoding a VHH including amino acid sequence SEQ ID NO: 1 that is expressed in the transgenic plant or plant tissue or plant cell, or progeny thereof. The at least one nucleotide sequence can include SEQ ID NO: 2. The receptor binding domain (RBD) of SARS-CoV-2 spike protein can include the sequence of SEQ ID NO: 3. The transgenic plant or plant tissue or plant cell, or progeny thereof, can be tobacco, carrot, and/or cabbage plants, e.g., plants of one or more of Nicotiana tabacum, Daucus carota, and Brassica oleracea.

Also described herein is a tissue culture produced from protoplasts or cells or callus tissue from the transgenic plant or plant tissue or plant cell, or progeny thereof, described herein. The protoplasts or cells or callus tissue are produced from a plant part such as, e.g., leaves, pollen, embryos, cotyledon, hypocotyl, meristematic cells, roots, root tips, pistils, anthers, flowers, stems, glumes and panicles. The tissue culture can be suspension culture. The tissue culture can include at least one bioreactor. The transgenic plant or plant tissue or plant cell, or progeny thereof, can be tobacco, carrot, and/or cabbage plants, e.g., plants of one or more of Nicotiana tabacum, Daucus carota, and Brassica oleracea.

Further described herein is a method for producing transgenic tobacco, carrot, or cabbage plants. The method includes the steps of: (a) stably transforming a plant or plant tissue or plant cell with at least one polynucleotide including at least one nucleotide sequence encoding a VHH that specifically binds to a receptor binding domain of SARS-CoV-2 spike protein under conditions such that the polynucleotide is integrated into the nuclear genome of the plant or plant tissue or plant cell; (b) selecting stably transformed cells, shoots, callus cells, embryos or seeds; (c) propagating transgenic plants from the stably transformed cells, shoots, callus cells, embryos or seeds selected in step (b); and (d) selecting transgenic plants propagated in step (c) having the at least one polynucleotide integrated within the nuclear genome and expressing the VHH. The method can further include the step of (e) transferring the transgenic plants selected in step (d) to soil conditions allowing for self-pollination to generate homozygous transgenic plant lines. In the method, step (a) typically includes Agrobacterium-mediated transformation. The transgenic tobacco, carrot, or cabbage plant can be one or more of: Nicotiana tabacum, Daucus carota, and Brassica oleracea. The at least one nucleotide sequence can include SEQ ID NO: 2, and the RBD of SARS-CoV-2 spike protein can include the sequence of SEQ ID NO: 3. In the method, the transgenic plants can be produced within 6-10 weeks (e.g., 6, 7, 8, 9, 10 weeks). In the method, at least step (c) can be performed in a greenhouse for large-scale production of the transgenic tobacco, carrot, or cabbage plants. In some embodiments, step (c) is performed additionally or alternatively in a field for large-scale production of the transgenic tobacco, carrot, or cabbage plants. The soil conditions can be in a greenhouse and/or a field.

Also described herein is a method for producing VHH that specifically binds to an RBD of SARS-CoV-2 spike protein. The method includes: (a) providing transgenic tobacco, carrot, or cabbage plants produced by the methods described herein; (b) propagating the transgenic tobacco, carrot, or cabbage plants in sterile conditions allowing continuous production of the VHH; and (c) isolating the VHH from the transgenic tobacco, carrot, or cabbage plants. In step (c), the VHH can be isolated from leaves of the transgenic tobacco, carrot, or cabbage plants. In the method, the step of stably transforming a plant or plant tissue or plant cell can include Agrobacterium-mediated transformation. The plant or plant tissue or plant cell, or the transgenic seeds, can be a transgenic tobacco, carrot, or cabbage plant or seed, e.g., one or more of Nicotiana tabacum, Daucus carota, and Brassica oleracea. In the method, the at least one nucleotide sequence can include SEQ ID NO: 2, and the RBD of SARS-CoV-2 spike protein can include (e.g., consist of) the sequence of SEQ ID NO: 3.

Still further described herein is a method for producing VHH that specifically binds to a RBD of SARS-CoV-2 spike protein. The method includes: (a) culturing transgenic plant tissue in a first callus medium including at least one plant hormone for initiation of primary compact callus, wherein the transgenic plant or plant tissue or plant cell, or progeny thereof, includes at least one polynucleotide integrated into the nuclear genome of the plant or plant tissue or plant cell, or progeny thereof, the at least one polynucleotide including at least one nucleotide sequence encoding a single variable domain of a VHH that specifically binds to the RBD of SARS-CoV-2 spike protein, wherein the at least one polynucleotide includes a nucleotide sequence encoding a VHH including amino acid sequence SEQ ID NO: 1 that is expressed in the transgenic plant or plant tissue or plant cell, or progeny thereof; (b) culturing the primary callus in the dark for a period sufficient to result in a primary compact callus; (c) transferring the primary compact callus to a second callus medium and propagating the primary compact callus until friable callus including loosely attached cells develops; (d) culturing the friable callus including loosely attached cells in liquid maintenance medium as a cell suspension; and (e) collecting cells from the cell suspension and isolating the VHH. In the method, step (d) can be performed in at least one bioreactor. The transgenic plant tissue can be at least one leaf segment. The at least one nucleotide sequence can include SEQ ID NO: 2, and the RBD of SARS-CoV-2 spike protein can include (e.g., consist of) the sequence of SEQ ID NO: 3. The transgenic plant tissue can be from one or more of: tobacco, carrot, and cabbage plants, e.g., from one or more of Nicotiana tabacum, Daucus carota, and Brassica oleracea.

The following terms or definitions are provided solely to aid in the understanding of the disclosure. Unless specifically defined herein, all terms used herein have the same meaning as they would to one skilled in the art of this disclosure.

As used herein, the terms “polypeptide,” “protein,” “peptide,” and “amino acid sequence” are used interchangeably, and refer to a polymeric form of amino acids of any length, which can include coded and non-coded amino acids, chemically or biochemically modified or derivatized amino acids, and polypeptides having modified peptide backbones. As used herein, amino acid residues will be indicated either by their full name or according to the standard three-letter or one-letter amino acid code.

As used herein, the terms “nucleic acid molecule,” “polynucleotide,” “polynucleic acid,” and “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural similarity between two macromolecules, particularly between two polypeptides or polynucleotides, from the same or different taxons, wherein the similarity is due to shared ancestry. Hence, the term “homologues” denotes so-related macromolecules having secondary and optionally tertiary structural similarity. For example, the spike (S) protein of SARS-CoV-2 has been found to be approximately 75% homologous to the SARS-CoV spike protein.

For comparing two or more polynucleotide sequences, the “(percentage of) sequence identity” between a first polynucleotide sequence and a second polynucleotide sequence may be calculated using methods known by the person skilled in the art, e.g., by optimally aligning the polynucleotide sequences and introducing gaps, if necessary, followed by dividing the number of nucleotides in the first polynucleotide sequence that are identical to the nucleotides at the corresponding positions in the second polynucleotide sequence in a comparison window by the number of positions in the comparison window (the window size), and multiplying by 100%. Optimal sequence alignment of two or more polynucleotide sequences over a comparison window can be obtained by using a known computer algorithm for sequence alignment such as NCBI Blast (Altschul et al., J. Mol. Biol. 1990 Oct. 5; 215 (3):403-410). Another example of an algorithm that is suitable for polynucleotide sequence alignments is the CLUSTALW program (J. D. Thompson, et al., Nucl. Acids Res. 1994 Nov. 11; 22 (22):4673-4680).

For comparing two or more polypeptide sequences, the “(percentage of) sequence identity” between a first polypeptide sequence and a second polypeptide sequence may be calculated using methods known by the person skilled in the art, e.g., by optimally aligning the polypeptide sequences and introducing gaps, if necessary, followed by dividing the number of amino acids in the first polypeptide sequence that are identical to the amino acids at the corresponding positions in the second polypeptide sequence in a comparison window by the number of positions in the comparison window (the window size), and multiplying by 100%. Optimal sequence alignment of two or more polypeptide sequences over a comparison window can be obtained by using a known computer algorithm for sequence alignment such as NCBI Blast (Altschul et al, J. Mol. Biol. 1990 Oct. 5; 215 (3):403-410). Another example of an algorithm that is suitable for polypeptide sequence alignments is the CLUSTALW program (J. D. Thompson et al., Nucl. Acids Res. 1994 Nov. 11; 22 (22):4673-4680). In determining the degree of sequence homology between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Conservative amino acid substitutions are counted as identities in order to calculate the percentage homology between two polypeptide sequences. Possible conservative amino acid substitutions will be clear to the person skilled in the art.

The terms “specifically bind” and “specific binding,” as used herein, generally refers to the ability of a polypeptide, in particular, an immunoglobulin, such as an antibody, or an immunoglobulin fragment, such as a VHH, to preferentially bind to a particular antigen (e.g., SARS-CoV-2 spike RBD) that is present in a homogeneous mixture of different antigens. Accordingly, an amino acid sequence as disclosed herein is said to “specifically bind to” a particular target when that amino acid sequence has affinity for, specificity for and/or is specifically directed against that target (or for at least one part or fragment thereof). The “specificity” of an amino acid sequence as disclosed herein can be determined based on affinity and/or avidity.

As used herein, the terms “inhibiting,” “reducing” and/or “preventing” may refer to (the use of) an amino acid sequence as disclosed herein that specifically binds to a target antigen of interest and inhibits, reduces and/or prevents the interaction between that target antigen of interest, and its natural binding partner. The terms “inhibiting,” “reducing” and/or “preventing” may also refer to (the use of) an amino acid sequence as disclosed herein that specifically binds to a target antigen of interest and inhibits, reduces and/or prevents a biological activity of that target antigen of interest, as measured using a suitable in vitro, cellular or in vivo assay. Accordingly, “inhibiting,” “reducing” and/or “preventing” may also refer to (the use of) an amino acid sequence as disclosed herein that specifically binds to a target antigen of interest and inhibits, reduces and/or prevents one or more biological or physiological mechanisms, effects, responses, functions pathways or activities in which the target antigen of interest is involved.

“Plant” as used herein, means live plants and live plant parts, including fresh fruit, vegetables and seeds. Also, the term “plant” as used herein encompasses whole plants, ancestors and progeny of the plants and plant parts, including seeds, shoots, stems, leaves, roots (including tubers), flowers, and tissues and organs.

The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

The terms “transgenic plant”, transgenic plant tissue” and “transgenic plant or plant tissue or plant cell, or progeny thereof” generally refer to plants or plant tissues or plant cells that have been genetically engineered to create plants with new characteristics (e.g., expressing VHH that specifically binds to the RBD of SARS-CoV-2 spike protein).

The terms “variable domain of a heavy-chain antibody” or “VHH” or “heavy chain variable domain of an antibody,” or “nanobodies” or “VNAR” or “sdAb” as used herein interchangeably, refer to the variable domain of the heavy chain of a heavy-chain antibody, which is naturally devoid of light chains, including but not limited to the variable domain of the heavy chain of heavy-chain antibodies of camelids or sharks. The total number of amino acid residues in a variable domain of a heavy-chain antibody or VHH can be in the region of about 110-130. It should however be noted that parts, fragments or analogs of a variable domain of a heavy-chain antibody are not particularly limited as to their length and/or size, as long as such parts, fragments or analogs retain (at least part of) the binding specificity of the original variable domain of a heavy-chain antibody from which these parts, fragments or analogs are derived from. Parts, fragments or analogs retaining (at least part of) the binding specificity of the original variable domain of a heavy-chain antibody from which these parts, fragments or analogs are derived from are also further referred to herein as “functional fragments” of a variable domain of a heavy-chain antibody. The VHH produced by the methods described herein do not have an amino acid sequence that is exactly the same as (i.e., as a degree of sequence identity of 100% with) the amino acid sequence of a naturally occurring VHH domain, such as the amino acid sequence of a naturally occurring VHH domain from a camelid or shark.

The term “expression” or “gene expression” means the transcription of a specific gene or specific genes or specific genetic construct. The term “expression” or “gene expression,” as used herein, refers to transcription of a polynucleotide or gene or genetic construct into structural RNA (rRNA, tRNA) or mRNA with or without subsequent translation of the latter into a protein. The process includes transcription of DNA and the processing of the resulting mRNA product. The term “encoding,” as used herein, refers to the transcription of a polynucleotide or gene, or genetic construct into structural RNA (rRNA, tRNA) or mRNA with the subsequent translation of the latter into a protein.

The term “introduction” or “transformation” as referred to herein encompass the transfer of an exogenous polynucleotide or chimeric gene (or expression cassette) into a host cell, irrespective of the method used for transfer.

Although transgenic plants, cell suspensions, tissue cultures and methods similar or equivalent to those described herein can be used in the practice or testing of the present invention, suitable transgenic plants, cell suspensions, tissue cultures and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a plasmid map showing the SARS-CoV-2 VHH-cMYC-12×His-tag sequence inserted into the pHEN6c plasmid.

FIG. 2 is a plasmid map showing the SARS-CoV-2 VHH-cMYC-12×His-tag sequence inserted into the pCDNA 3.4 plasmid (Thermo Fisher Scientific, Boston, MA) using XbaI and AgeI restriction sites.

FIG. 3 is a map of vector pBI121-VHH that was used for plant transformation.

FIG. 4 is a graph showing the ability of bacteria and plant cells-derived SARS-CoV-2 VHH antibody to bind SARS-COV2 spike protein. Means±SD are shown.

FIG. 5 top is the sequence (SEQ ID NO: 13) of a PCR product in a PCR analysis confirming incorporation of the VHH-antibody gene into plant genomes (the sequence of primers is highlighted in bold). FIG. 5 bottom is a schematic of the structure of VHH PCR primers and product.

### DETAILED DESCRIPTION

The most popular method for producing monoclonal antibodies is based on Chinese hamster ovary (CHO) cells, which can be grown in large bioreactors. However, this process is slow and can take many months to determine productive CHO cell culture and scale it up in large bioreactors. In addition, the process of growing these cell cultures, as well as the research to identify and develop the most effective antibodies can make such therapies extremely expensive. Plants have become a prospective replacement bioreactor for currently available production systems to manufacture biopharmaceuticals. Moreover, plants offer several advantages as a mAb production system, such as the lack of human pathogens, relatively low-cost manufacturing, ease of production scale-up, and are inherently safe because no human pathogens can replicate in plants. As described herein, single-domain monoclonal antibodies against SARS-CoV-2 were developed using a plant system, and plant-derived antibodies were compared with those produced in bacterial and mammalian cell systems. Three VHH-containing vectors capable of transfecting bacteria, mammalian, and plant cells were developed based on plasmids pHEN6c, pcDNA™ and pBI121, respectively. In this work, WK6 bacteria and EXPI293F™ mammalian cells were used to produce the SARS-CoV-2 VHH antibody. The produced proteins were extracted based on the affinity of 12×-Histag to the Ni-NTA resin column, and their production and purity were confirmed using SDS-Page and Western blot analysis. The experimental results demonstrated the ability of produced antibodies to bind to SARS-COV2 spike protein. In the experiments described herein, stable transgenic tobacco plants constantly expressing VHH antibodies were produced by the inventive method described herein very quickly—approximately in 2 months—compared to a longer span of months required for other production systems. Transgenic plants producing antibodies, vaccines, and other recombinant proteins via molecular farming are considered as bioreactors or factories; cultivation of them can be expanded to an agricultural scale and has the potential to fulfill emergency demands, such as in the present situation of the COVID-19 pandemic. Described herein are methods for producing VHH that specifically binds to the RBD of SARS-CoV-2 spike protein, and transgenic plants, plant parts, plant cells, plant tissue, and seeds that express the VHH that specifically binds to the RBD of SARS-CoV-2 spike protein.

### VHH Antibodies (Nanobodies)

Camelid antibodies have been studied as alternatives to conventional human antibodies (Könning et al. Current Opinion in Structural Biology 2017, 45, 10-16; Danis et al. Mol Ther 2022, 30, 1484-1499; Ezzikouri et al. J Biomol Struct Dyn 2022, 40, 3129-3131). In addition to conventional heterotetrameric antibodies, serum in Camelids (dromedaries, Bactrian camels, and llamas), as well as cartilaginous fish, contains unique single N-terminal domain functional heavy (1H)-chain antibodies. The recombinant single variable domain camelid-derived heavy chain antibody fragments (VHH, also referred to as “nanobody” and often abbreviated as VHH, VNAR, sdAb) possess exclusive properties and are well known in the art (e.g., Mehdi Arbabi-Ghahroudi Int J Mol Sci. 2022 May; 23(9): 5009; US Patent Pub. No. 20210340556; WO 04/041867, WO 04/041862, WO 04/041865, WO 04/041863, WO 04/062551, all incorporated by reference herein). The VHH can bind to antigens without requiring domain pairing and can also recognize antigenic sites that are normally not recognized by conventional antibodies. Single-domain antibodies consist of a single monomeric variable antibody domain and do not contain the light chain and CH domain of the conventional Fab region's heavy chain. Unique characteristics of VHH, such as small size, high affinity/specificity to antigens, stability and relatively low production cost, make them an attractive alternative to traditional antibodies as well as antibody fragments in therapeutic applications (Könning et al. Current Opinion in Structural Biology 2017, 45, 10-16; Jovc̆evska and S. Muyldermans BioDrugs 2020, 34, 11-26; S. Muyldermans Annu Rev Biochem 2013, 82, 775-797). In addition, these small antibodies benefit from improved penetration to sites of infection. The VHH described herein may be efficient for the treatment of respiratory diseases and particularly for COVID-19 due to the possibility of alternative routes of administration, especially nasal and inhalation routes. For example, these antibodies can be nebulized and administered via an inhaler directly to infected lungs (Haga et al. PLoS Pathog 2021, 17, e100954). Consequently, VHH specific to COVID-19 spike protein can form a basis for an effective and innovative approach to treating infection by the SARS-CoV-2 virus.

Transgenic Plants. Plant Tissues, Transgenic Plant Cells, Progeny Thereof

Described herein are transgenic plants, plant tissues, plant cells, and progeny thereof, from which VHH that specifically binds to the RBD of SARS-CoV-2 spike protein can be isolated and purified. A transgenic plant or plant tissue or plant cell, or progeny thereof can be a tobacco, carrot, or cabbage plant, e.g., Nicotiana tabacum, Daucus carota, or Brassica oleracea. The transgenic plant or plant tissue or plant cell, or progeny thereof includes at least one polynucleotide integrated into the nuclear genome of the plant or plant tissue or plant cell, or progeny thereof. The at least one polynucleotide includes at least one nucleotide sequence encoding a VHH that specifically binds to the RBD of SARS-CoV-2 spike protein. In embodiments the at least one polynucleotide includes a nucleotide sequence encoding a VHH comprising amino acid sequence SEQ ID NO: 1 that is expressed in the transgenic plant or plant tissue or plant cell, or progeny thereof. SEQ ID NO: 1 is the VHab8 amino acid sequence having GenBank Reference No. MT943599. This amino acid sequence, with C-terminal tags c-Myc and His-tag, is shown in Table 5 as SEQ ID NO: 4. VHHs that specifically bind to an RBD of SARS-CoV-2 spike protein may, for example, include, consist essentially of, or consist of an amino acid sequence that is at least 60%, 61%, 62%, 63%, 64%, 65%, 66%, 67%, 68%, 69%, 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1. Variants and analogs within are also contemplated. VHHs that specifically bind to the RBD of SARS-CoV-2 spike protein as described herein do not include an Fc region.

Within at least one polynucleotide integrated into the nuclear genome of the plant or plant tissue or plant cell, or progeny thereof, at least one nucleotide sequence can include SEQ ID NO: 2, which is a codon-optimized VHab8 nucleotide sequence for plant expression. There are differences between plant-optimized sequence and GenBank MT943599 sequence: they are 74.73% identical and the difference is 25.27% (this difference is due to optimization of the codon for plant expression and the translated amino acid sequences are identical). In embodiments, the at least one nucleotide sequence can or may, for example, include, consist essentially of, or consist of a nucleotide sequence that is at least 60%, 61%, 62%, 63%, 64%, 65%, 66%, 67%, 68%, 69%, 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2. Any polynucleotide or nucleotide sequence that is codon-optimized for plant expression and that encodes a VHH that specifically binds to the RBD of SARS-CoV-2 spike protein is contemplated. As used herein, the wording “at least one polynucleotide including at least one nucleotide sequence encoding a VHH” and “at least one polynucleotide encoding a VHH” may be used interchangeably. In embodiments, the transgenic plant or plant tissue or plant cell or progeny thereof may not include any polynucleotide (comprising at least one sequence) encoding a light chain of an antibody, such as an immunoglobulin light chain.

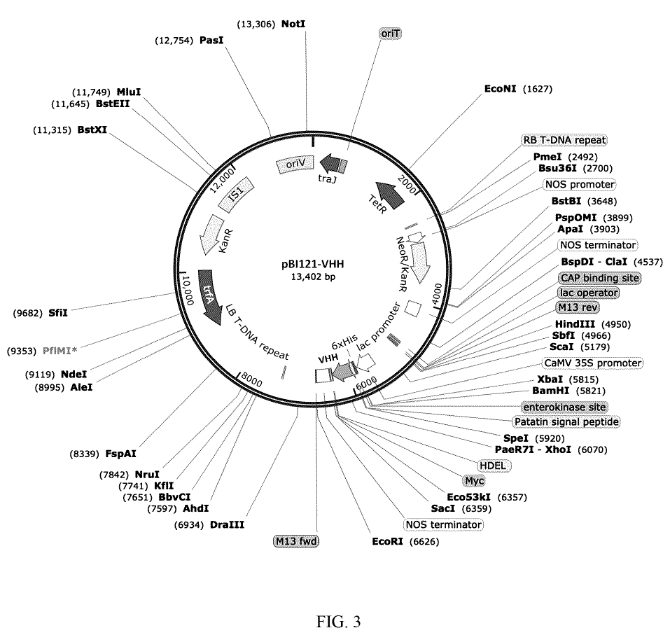

The nucleic acid sequences capable of encoding a VHH in a transgenic plant or plant tissue or plant cell as defined herein can be in the form of a vector or a genetic construct or polynucleotide. As used herein, the terms “genetic construct” and “nucleic acid construct” are used interchangeably. The nucleic acid sequences as disclosed herein may be synthetic or semi-synthetic sequences, nucleotide sequences that have been prepared by PCR using overlapping primers, or nucleotide sequences that have been prepared using techniques for DNA synthesis known per se. The genetic constructs of the disclosure can be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the disclosure may be in the form of a vector, such as, for example, a plasmid, a viral vector or transposon. In particular, the vector may be an expression vector, i.e., a vector that can provide for expression in vitro and/or in vivo in a suitable host cell, host organism and/or expression system. For example, the vector shown in FIG. 3 can be used for plant transformation via Agrobacterium-mediated transformation. This vector includes a synthetic cDNA encoding the VHH gene under the control of Cauliflower Mosaic Virus (CaMV-35) promoter suitable for expression in plant cells. Plant-produced VHH specifically binds to the RBD of SARS-CoV-2 spike protein. Any suitable regulatory or control sequences can be included in a vector or plasmid used in the methods described herein. Generally, a regulatory or control nucleic acid sequence is operatively linked to, or associated with, a nucleic acid sequence that codes for an mRNA, such that the regulatory nucleic acid sequence is able to regulate transcription or expression of the associated nucleic acid coding sequence when introduced into a cell such as a plant cell. In embodiments, the at least one polynucleotide integrated into the nuclear genome includes a promoter suitable for expression in plants (e.g., CaMV-35S promoter), a plant tissue or plant cell specific promoter, or an inducible promoter, and a 3′ end region including transcription termination and polyadenylation signals functioning in cells of the plant. Another example of a genetic control or regulatory element that can be included is an enhancer. The terms “plant promoter” or “promoter suitable for expression in plants” as used herein refers to a nucleic acid sequence comprising regulatory or control elements, which mediate the expression of a coding sequence in plant cells. For expression in plants, the nucleic acid molecule is linked operably to or includes a suitable promoter that expresses the gene at the right point in time and with the required spatial expression pattern. The term “operably linked” as used herein refers to a functional linkage between the promoter sequence and the gene of interest, such that the promoter sequence is able to initiate transcription of the gene of interest.

The RBD of SARS-CoV-2 spike protein that the VHH specifically binds to can include the amino acid sequence of SEQ ID NO: 3. This sequence is recombinant human coronavirus SARS-CoV-2 Spike Glycoprotein RBD sequence, isolate Wuhan-Hu-1, commercially available from Abcam (Boston, MA). This sequence includes a His tag at the C-Terminus. In the experiments described below, VHH produced by transgenic plants and transgenic cells were shown to bind to the sequence of SEQ ID NO: 3. In other embodiments, a VHH as described herein specifically binds to the RBD of a SARS-CoV-2 variant, e.g., a variant of interest or a variant of concern. In such embodiments, once the amino acid sequence for the RBD of that variant is made available, a plant or plant tissue or plant cell can be stably transformed with a polynucleotide including a nucleotide sequence encoding a VHH that specifically binds to the variant's RBD according to the methods described herein. Accordingly, the VHH described herein can be used for treatment and/or prophylaxis of SARS-CoV-2 infections caused by novel emerging SARS-CoV-2 variant strains.

Also described herein is a tissue culture produced from protoplasts or cells or callus tissue from a transgenic plant or plant tissue or plant cell, or progeny thereof, as described herein. In embodiments, the protoplasts or cells or callus tissue are produced from a plant part such as, for example, leaves, pollen, embryos, cotyledon, hypocotyl, meristematic cells, roots, root tips, pistils, anthers, flowers, stems, glumes and panicles. The transgenic plant or plant tissue or plant cell, or progeny thereof can be a tobacco, carrot, or cabbage plant, e.g., Nicotiana tabacum, Daucus carota, or Brassica oleracea. In embodiments, the tissue culture is a suspension culture. In such embodiments, the tissue culture can include one or more (e.g., 1, 2, 3, 4, 5, 6, 7, 8, 9, 10, etc.) bioreactors. Methods of growing plant cells in suspension culture, including in bioreactors, are well known in the art. See, for example, Marchev et al. Critical Reviews in Biotechnology, 2020, 40, 443-458; Wu et al. Plant Communication 2021, 13, 2(5), 100235. A tissue culture as described herein can include any suitable culture medium. Examples of suitable culture media include propagation medium, selection medium, and maintenance medium. An example of a maintenance medium for callus is MSCT medium which was used in the experiments described below. This MSCT medium includes basic MS basal medium supplemented with 3% sucrose, 0.7% agar, 2 mg/L 2,4-D, 200 mg/L timentin, and 100 mg/L kanamycin. Table 4 below includes examples of media for tissue culture.

Methods for the generation of transgenic plants including recombinant DNA techniques are well-known in the art. Recombinant methodologies generally involve inserting a DNA molecule expressing an amino acid sequence, protein, or polypeptide of interest into an expression system to which the DNA molecule is heterologous (i.e., not normally present in the host). The heterologous DNA molecule is inserted into the expression system or vector in proper sense orientation and correct reading frame. The vector contains the necessary elements for the transcription and translation of the inserted protein-coding sequences. For example, the transcription of DNA is dependent upon the presence of a promoter. Regardless of the specific regulatory sequences employed, the DNA molecule is cloned into the vector using standard cloning procedures in the art, as described by Sambrook et al, Molecular Cloning: A Laboratory Manual, Cold Springs Laboratory, Cold Springs Harbor, N.Y. (1989). Once the isolated DNA molecule encoding the protein has been cloned into an expression system, it is ready to be incorporated into a host cell. Such incorporation can be carried out by the various forms of transformation, depending upon the vector/host cell system.

In embodiments, a method for producing transgenic tobacco, carrot, or cabbage plants includes the steps of: (a) stably transforming a plant or plant tissue or plant cell with at least one polynucleotide comprising at least one nucleotide sequence encoding a VHH that specifically binds to the RBD of SARS-CoV-2 spike protein under conditions such that the polynucleotide is integrated into the nuclear genome of the plant or plant tissue or plant cell; (b) selecting stably transformed cells, shoots, callus cells, embryos or seeds; (c) propagating transgenic plants from the stably transformed cells, shoots, callus cells, embryos or seeds selected in step (b); and (d) selecting transgenic plants propagated in step (c) having the at least one polynucleotide integrated within the nuclear genome and expressing the VHH. In embodiments of the method, the nucleotide sequence encodes a VHH including amino acid sequence SEQ ID NO: 1 that is expressed in the transgenic plant or plant tissue or plant cell, or progeny thereof, the at least one nucleotide sequence includes SEQ ID NO: 2, and the RBD of SARS-CoV-2 spike protein includes the sequence of SEQ ID NO: 3. The transgenic tobacco, carrot, or cabbage plant can be Nicotiana tabacum, Daucus carota, and Brassica oleracea. The method can further include transferring the transgenic plants selected in step (d) to soil conditions (e.g, in a greenhouse) allowing for self-pollination to generate homozygous transgenic plant lines. The step of stably transforming a plant or plant tissue or plant cell with at least one polynucleotide typically includes Agrobacterium-mediated transformation. Any suitable method of Agrobacterium-mediated transformation can be used. For example, Agrobacterium-mediated plant transformation using disarmed Ti plasmids is well known in the art. See, e.g., Kiyokawa et al., Appl Environ Microbiol. 2009 April; 75(7): 1845-1851.

The transgenic plants, plant tissues, plant cells, and progeny thereof include at least one polynucleotide as described herein stably integrated into their nuclear genome. Upon stable integration of nucleic acids into plant cells, typically only a subset of the cells takes up the foreign DNA and integrates it into its genome, depending on the expression vector used and the transfection technique used. To identify and select these integrants, a gene coding for a selectable marker (e.g., the nptII gene for kanamycin selection) is usually introduced into the host cells together with the gene of interest (on the same vector, or in a separate vector). Cells that have been stably transfected with the introduced nucleic acid can be identified, for example, by selection (for example, cells that have integrated the selectable marker survive whereas the other cells die).

Plant tissue or plant cells capable of subsequent clonal propagation, whether by organogenesis or embryogenesis, may be transformed with at least one polynucleotide as described herein and a whole plant regenerated therefrom. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art. In embodiments of the method, transgenic plants are produced within about 6-10 weeks (e.g., 5.0, 5.5, 6, 6.5, 7, 7.5, 8, 8.5, 9, 9.5, 10, 10.5, 11 weeks). The step of propagating transgenic plants from the stably transformed cells, shoots, callus cells, embryos, or seeds selected can be performed in a greenhouse for large-scale production of the transgenic tobacco, carrot, or cabbage plants. In other embodiments, the step of propagating transgenic plants from the stably transformed cells, shoots, callus cells, embryos or seeds selected can be performed in a field for large-scale production of the transgenic tobacco, carrot, or cabbage plants.

### Production of VHH in Plants

Methods of producing VHH that specifically bind to the RBD of SARS-CoV-2 spike protein are described herein. Due to strong market demand, the production of anti-COVID-19 antibodies requires industrial-scale production. Traditional large-scale manufacturing processes are based on mammalian cell systems, which require high initial investments and substantial production costs. Additionally, the high risk of contamination with viruses, prions, oncogenic DNA and other pollutants requires additional efforts leading to a further increase in cost. Other complications caused by bioreactors such as development time, scalability, yield, and authenticity make the development of alternative production platforms more compelling. Unlike most full-length mAbs which can only be efficiently produced in mammalian cells, nanobodies can be produced in bacterial systems, because they do not require glycosylation. E. coli's rapid growth in inexpensive media and well-establish genetics make it a popular system for nanobody expression. However, bacterial systems have several disadvantages such as the formation of inclusion bodies, protease and endotoxin contamination. Therefore, there is an urgent need for alternative production platforms. Plants offer unique advantages as an antibody production system, such as the lack of human/animal pathogens or bacterial toxins, relatively low-cost manufacturing, and ease of production scale-up (Pogrebnyak et al. Plant Sci 2006, 171, 677-685; Brodzik et al. Proc Natl Acad Sci USA 2006, 103, 8804-8809). Unlike other expression systems, plant systems can be implemented in different forms, including the cultivation of whole plants producing recombinant therapeutics in fields, transient expression based on viral vectors, and plant cell suspension culture growing under aseptic conditions.

In embodiments, a method for producing VHH that specifically binds to the RBD of SARS-CoV-2 spike protein includes the steps of: providing transgenic tobacco, carrot, or cabbage plants produced by the methods described herein; propagating the transgenic tobacco, carrot, or cabbage plants in sterile conditions allowing continuous production of the VHH; and isolating the VHH from the transgenic tobacco, carrot, or cabbage plants. In the method, the step of stably transforming a plant or plant tissue or plant cell typically includes Agrobacterium-mediated transformation. The VHH can be isolated from the transgenic plant or plant tissue or plant cell or transgenic seed, and purified, using any suitable methods known in the art. In embodiments, the VHH is isolated primarily from the leaves of transgenic plants. The plant or plant tissue or plant cell, or the transgenic seeds typically are a transgenic tobacco, carrot, or cabbage plant or seed. For example, a transgenic tobacco, carrot, or cabbage plant or seed can be Nicotiana tabacum, Daucus carota, or Brassica oleracea. In these methods, the VHH produced can include SEQ ID NO: 1, the at least one nucleotide sequence can include SEQ ID NO: 2, and the receptor binding domain of SARS-CoV-2 spike protein can include the sequence of SEQ ID NO: 3.

### Production of VHH in Suspension Culture

Another form of plant bioreactor is cell suspension, which produces VHH antibodies in sterile conditions with a lack of human/animal pathogens, relatively low-cost manufacturing, and ease of production scale-up. Plant cell cultures are based on plant cells growing in a fully controlled environment, and offer the advantage of producing proteins in bioreactors under the conditions of the good manufacturing practice. Suspension cell cultures have the same advantages of sterility and containment similar to other cell culture expression systems. The ability to use low-cost plant growth medium represents an additional advantage over expensive mammalian medium. Similar plant biotechnology approaches can be used for producing monoclonal neutralizing antibodies against other types of coronaviruses.

In embodiments, a method for producing VHH that specifically binds to the RBD of SARS-CoV-2 spike protein includes the steps of: (a) culturing transgenic plant tissue prepared by the methods described herein in a first callus medium including at least one plant hormone for initiation of primary compact callus; (b) culturing the primary callus in the dark for a period sufficient to result in a primary compact callus; (c) transferring the primary compact callus to a second callus medium and propagating the primary compact callus until friable callus including loosely attached cells develops; (d) culturing the friable callus including loosely attached cells in liquid maintenance medium as a cell suspension; and (e) collecting cells from the cell suspension and isolating the VHH. In the method, any suitable transgenic plant tissue can be used, e.g., a leaf segment(s). Any suitable first callus medium that includes at least one plant hormone (e.g., a cytokinin such as BAP, auxins such as NAA and 2,4-D, etc.) can be used, for example MSCIT medium with 3% sucrose, 0.7% agar, 1 mg/L 2,4-D, 0.5 mg/L NAA, 0.5 mg/L BAP, 300 mg/L timentin and 100 mg/L kanamycin. In the step of culturing the primary callus in the dark, the period of sufficient time is typically about 2-3 weeks (e.g., 12, 13, 14, 15, 16, 17, 18, 19, 20, 21, 22 days). In the step of transferring the primary compact callus to a second callus medium, any suitable second callus medium can be used. A second callus medium typically includes at least 1 plant hormone (e.g., 2,4-D). A second callus medium can include not only, for example, auxin 2,4-D, but another auxin, such as NAA. In some cases, the second callus medium can contain both types of growth stimulators: auxins (2,4-D, NAA) and cytokinins (BAP, Kinetin) while at a high ratio of auxin-to-cytokinin. Determining when friable callus including loosely attached cells has developed typically involves about 3 weeks. The friability is a morphological feature, where the callus presents a soft texture and loosely associated cells. In the step of culturing the friable callus including loosely attached cells in a liquid maintenance medium as a cell suspension, any suitable maintenance medium can be used. An example of a suitable maintenance medium is any that includes 2,4-D at a suitable concentration, e.g., about 2 mg/L). For example, MSST medium contain MS basal medium supplemented with 3% sucrose, 2 mg/L 2,4-D, 100 mg/L timentin and 100 mg/L kanamycin. The step of culturing the friable callus including loosely attached cells in a liquid maintenance medium as a cell suspension provides for permanent, continuous production of VHH. Cells producing the VHH can be collected using any suitable method. VHH can be isolated from the collected cells, and purified, using any suitable methods known in the art.

In embodiments of a method for producing VHH that specifically binds to the RBD of SARS-CoV-2 spike protein, the step of culturing the friable callus in a liquid maintenance medium as a cell suspension is performed in one or more bioreactors. Methods of growing plant cells in suspension culture, including in bioreactors, are well known in the art. See, for example, Raven et al. Biotechnol. Bioeng 2015, 308-321.

### EXAMPLES

The present invention is further illustrated by the following specific examples. The examples are provided for illustration only and should not be construed as limiting the scope of the invention in any way.

### Example 1—Plant-Derived Single Domain COVID-19 Antibodies

The present investigation was aimed at developing single-domain monoclonal antibodies against SARS-CoV-2 using mammalian, bacterial, and plant-based expression systems while comparing the resulting products based on their characteristics, affinity, and stability to propose a suitable method for the production of VHH nanobodies for the treatment of SARS-CoV-2 infection. The specific aim of this investigation was to produce transgenic plants and plant cell suspensions that express SARS-CoV-2 antibodies targeting the receptor-binding domain on the spike protein. For the production of such antibodies, the human heavy chain variable domain VHH that shows potent neutralization activity and specificity against SARS-CoV-2 in vitro and in animal models was focused on.

### Materials and Methods

Preparation of Chemically Competent E. coli Cells for VHH Cloning

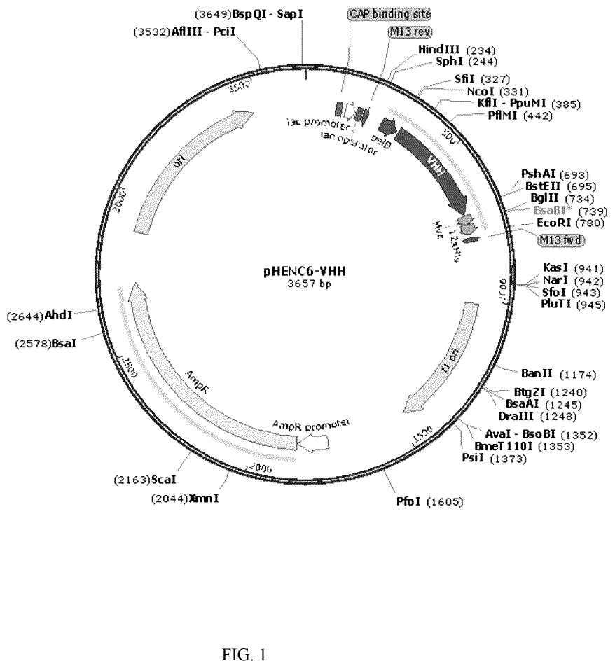

Using standard cloning techniques, the DNA sequences encoding VHH for SARS-CoV-2 (GenBank, MT943599) (Li et al. Cell 2020, 183, 429-441 e416) were designed, codon-optimized for bacterial expression, and cloned into the pHEN6c bacterial expression vector with a C-terminal 12×His-tag using NcoI and EcoRI restriction sites. The amino acid sequence of used SARS-CoV-2 VHH and the structure of the plasmid with an inserted sequence is shown in Table 1 and FIG. 1, respectively. Cellular Myelocytomatosis (c-Myc) and protein affinity 12×His-tag sequences were incorporated in the vectors allowing detection and purification of the resulting VHH protein. The cloned vectors were transformed into WK6 Chemically Competent E. coli Cells (Thermo Fisher Scientific, Boston, MA) using the recommended transformation protocol. Briefly, 5 μL of WK6 cells were spread on LB agar (without antibiotic) plates for 14 h or overnight. Then, colonies of the overnight culture were inoculated into 100 mL LB media, and the culture was grown to the optical density at 600 nm of 0.6 units (after 4 h). After that, the cells were incubated on ice for 10 min. Following this step, the bacteria were pelleted at 4000 g for 20 min at 4° C. The pellet was resuspended in 50 mM CaCl₂ and placed on ice for 30 min. Next, the cell suspensions were centrifuged as explained above, and pellets were dissolved in 15 mL CaCl₂) (50 mM). At this stage, the plate was centrifuged at 4° C. at 4000 g for 10 min, and the supernatant was discarded. Again, the pellet was resuspended in 1 mL ice-cooled 50 mM CaCl₂, and 50 μL aliquots were prepared and stored at −80° C. for further experiments.

TABLE 1
The amino acid sequence of the SARS-COV-2
VHH expressed by E. coli cells transformed
with the pHENC6-VHH plasmid shown in FIG.
1. The bolded letters show the position of
the added c-Myc, and the underlined letters
show the position of the 12x His-tag.

| Vector Name | Vector Sequence |
|---|---|
| pHENC6-VHH- | EVQLVESGGGLVQPGGSLRLSCAASGFTFDDYAM |
| c-Myc-12xHis- | SWVRQAPGKGLEWIGRMYNNGRTSYNPSLKSLVT |
| tag | ISRDNSKNTLYLQMNSLRAEDTATYYCARDNLGY |
| | RPSENLYGMDVWGQGTTVTVSSEQKLISEEDLHH |
| | HHHHHHHHHH (SEQ ID NO: 5) |

Competent E. coli Cells Transformation

WK6 Chemically Competent E. coli Cells transformations were performed as follows; 50 μL of thawed competent bacteria and 5 μL (1 pg-100 ng) of pDNA or pHEN6c-VHH sample (2 μL stock pDNA+18 μL CaCl₂) were added to the pre-chilled tube and pipetted gently. Then, the suspensions were kept on ice for 30 min, and after that, the mixture was heated at 42° C. for 45 sec. Following the heat-shock step, the tubes were kept on ice for another 2 min. Then 950 μL of Recovery Medium (SOC, Sigma Aldrich, St. Louis, MO) was added, and the suspension was incubated in a shaker at 37° C. for 1 h. Next, 10 μL of the transformation was spread on prewarmed selection plates (containing 100 μg/mL Carbenicilin), and the plates were incubated overnight at 37° C. The next day, 1l colonies were selected and expanded in 5 mL of LB Broth (containing 100 μg/mL Carbenicilin), and the peptide expression was induced by IPTG (0.4 mM) at the OD 600 of three units. Then the colonies were checked for V_{H} ab8 protein production using SDS-PAGE (NuPAGE™ 4 to 12%, Bis-Tris, 1.0 mm, Mini Protein Gel) and PageBlue™ Protein staining (Thermo Fisher Scientific, Boston, MA) based on manufacturer protocol. The suitable colony was expanded in 500 mL LB Broth (containing 100 μg/mL Carbenicilin), and the peptide expression was induced by IPTG (0.4 mM) at the optic density at 600 nm of three units. Bacterial cells were grown for 4 h post-induction. Then the bacterial solution was centrifuged (6000×g, 10 min). The resulting bacterial pellet was stored at −80° C. for further experiments.

### Mammalian Cell Culture Conditions

EXPI293F™ cells were grown in Expi293 medium (Thermo Fisher Scientific, Boston, MA) at 37° C., 8% CO₂ atmosphere in plastic Corning™ Cell Culture Treated Flasks (Thermo Fisher Scientific, Boston, MA). A CO2 Resistant orbital shaker (Thermo Fisher Scientific, Boston, MA) was used for the incubation. The cells were split to 0.3×10⁶ viable cells/mL when they reached a 3×10⁶/mL density. Cell density was determined with a Scepter cell counter (Millipore, Burlington, MA) during the expansion and maintenance phase. One day before transfection, the medium was replaced by spinning down the cells at 300×g for 5 min at room temperature and then carefully decanting or pipetting away all cell media before resuspending the cells in the prewarmed fresh Expi293 medium.

### Mammalian Cell Transfection

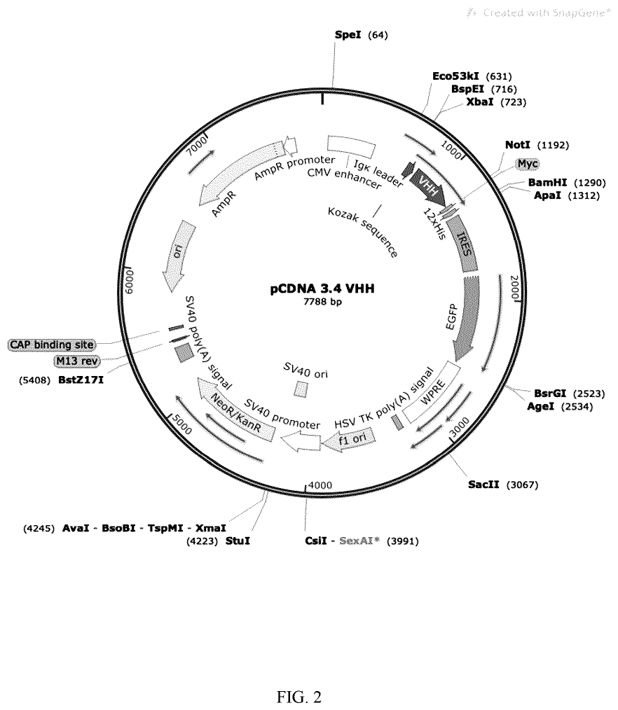

On transfection day, the cells were spun at 300×g for 5 min at room temperature. The medium was removed by aspiration. Then, the cells were resuspended in the culture medium at a final concentration of 2×10⁶/mL. The cells were transfected with a pcDNA3.4-VHH mammalian expression vector with a C-terminal 12×His-tag by using Expi293™ Expression System Kit (Thermo Fisher Scientific, Boston, MA) following the manufacturer's protocol. The amino acid sequence of used SARS-CoV-2 VH ab8 VHH and the structure of the plasmid with an inserted sequence are shown in Table 2 and FIG. 2, respectively. After 24 h incubation at 37° C., 8% CO₂ atmosphere, the cells were transferred to a larger flask and diluted with prewarmed Expi293™ medium to a concentration of 0.3×10⁶/mL. The transfected cells were incubated for 7-10 days at the condition mentioned above, and the cell viability was regularly checked every second day.

TABLE 2
The amino acid sequence expressed by the
SARS-COV-2 VHH recombinant vectors pCDNA
3.4 VHH of FIG. 2. The bolded letters show
the position of the added c-Myc, and the
underlined letters show the position of the
12x His-tag. Sequences of added linkers are
lowercase.

| Vector Name | Vector Sequence |
|---|---|
| pCDNA 3.4 VHH- | EVQLVESGGGLVQPGGSLRLSCAASGFTFDDYA |
| c-Myc-12xHis- | MSWVRQAPGKGLEWIGRMYNNGRTSYNPSLKSL |
| tag | VTISRDNSKNTLYLQMNSLRAEDTATYYCARDN |
| | LGYRPSENLYGMDVWGQGTTVTVSSaaaEQKLI |
| | SEEDLnsvadHHHHHHHHHHHH (SEQ ID |
| | NO: 6) |

Purification of the SARS-CoV-2 VHH from Bacterial and Mammalian Cells

For purification of the SARS-CoV-2 VHH from WK6 Chemically Competent E. coli cells, the bacterial pellet was lysed using the lysis buffers A and B (Table 3). For every gram of bacterial pellet, 10 mL of lysis buffer A and 15 mL of lysis buffer B were used. The slurry was vigorously stirred at 4′C for 1 h, centrifuged at 20,000 g (4° C.) for 1 h, followed by passing through a 0.2 μL PES membrane filter column (VWR, Radnor, PA) using a vacuum-driven filtration. In the next step, Ni-NTA resin (1 mL resin per 30 g of the pellet) was added to the filtered suspension, and the sample was incubated with the Ni-NTA resin for 1 h at room temperature while rocking gently. The mix was then passed through the Econo-Pac® Chromatography Columns (Bio-Rad, Hercules, CA) followed by washing the column with 50 mL of Imidazole 10 mM in the first step and then 50 mL of Imidazole 30 mM. For purification of the SARS-CoV-2 VHH from EXP1293Fr cells, the cells were spun at 300×g for 5 min. Then the supernatant was vacuum filtered using a 0.2 μL PES membrane filter column (VWR, Radnor, PA). In the next step, 500 μL Ni-NTA resin was added to the filtered suspension, and the sample was incubated with the Ni-NTA resin for 1 h at room temperature while rocking gently. The mix was then passed through the Econo-Pac® T Chromatography Columns (Bio-Rad, Hercules, CA). The VHH antibody was eluted with 500 μL of the elution buffer and then mixed with 500 μL of glycerol and stored at −20° C. for further experiments.

TABLE 3
The compositions of lysis and elution buffers
(pH = 7) for protein purification.

| Reagents | Lysis Buffer A | Lysis Buffer B | Elution Buffer |
|---|---|---|---|
| NaH2PO4 | — | — | 20 mM |
| Tris | 24.22 g/L | 8.1 g/L | — |
| NaCl | — | — | 500 mM |
| Imidazole | — | — | 500 mM |
| EDTA | 0.2 g/L | 0.05 g/L | — |
| Sucrose | 170 g/L | 43 g/L | — |

Sodium Dodecyl-Sulfate Polyacrylamide Gel Electrophoresis (SDS-PAGE) and Western Blot

Before SDS-PAGE, VHH was quantitated using a BCA Protein Assay Kit (Thermo Fisher Scientific, Boston, MA) based on manufacturer protocol. To analyze the VHH antibody's purity, SDS-PAGE (4-20% Mini-PROTEAN® TGX™ Precast Protein Gels, Bio-Rad, Hercules, CA) and PageBlue™ Protein staining (Thermo Fisher Scientific, Boston, MA) was used based on manufacturer protocol. To confirm the expression of the VHH antibody, the Western blot was used. Briefly, 100 μg of samples were denatured at 95° C. for 10 min and transferred to a 4-20% Mini-PROTEAN® TGX™ Precast Protein Gels (Bio-Rad, Hercules, CA), and electrophoresis was performed at 120 V for 60 min. Then, the band was transferred to a 0.2 μm cellulose nitrate membrane (Thermo Fisher Scientific, Boston, MA). The filters were incubated in 5% BSA TBST at room temperature for 2 h with a shaker to avoid unspecific binding. Subsequently, filters were incubated with DyLight™ 680 Conjugated Affinity Purified anti-His-tag antibody (Rockland Inc, Pottstown, PA) at 1:10000 dilution, at 4° C. overnight. Afterward, the immunoreactive bands were detected by ChemiDoc MP Imaging System (Bio-Rad, Hercules, CA). A quantitative Western blot was performed by a regression analysis of a calibration curve created using purified bacteria VHH protein in a 2× serial dilution. The concentration of plant-derived VHH protein was determined by analyzing band intensity.

### The Enzyme-Linked Immunosorbent Assay (ELISA)

ELISA was performed to evaluate the ability of the VHH antibody to bind to the SARS-COV2 spike protein. For this purpose, 100 μL of 1 μg/mL recombinant human coronavirus SARS-CoV-2 Spike Glycoprotein RBD (Abcam, Boston, MA) was coated onto an Immuno-Maxi Absorbance 96-well plate (Thermo Fisher Scientific, Boston, MA) and incubated overnight at 4° C. Each well was washed three times with phosphate-buffered saline (PBS) plus 0.1% Tween (PBS-T) and subsequently blocked by 2% bovine serum albumin (BSA, VWR, Radnor, PA) in PBS-T for 2 h at room temperature. Then, different concentrations of VHH ranging from 0 to 100 μg/mL were added to each SARS-CoV-2 Spike Glycoprotein RBD-coated well and incubated for 30 min at room temperature. Each well was washed three times by PBS-T and then incubated with 100 μL of the HRP-conjugated Anti-c-Myc antibody (Abcam, Boston, MA) for 60 min at room temperature. Samples were washed six times by PBS-T, followed by the addition of 50 μL 3,3′,5,5′-tetramethylbenzidine (TMB), and incubation for 15 min at room temperature. Finally, the reaction was stopped by 50 μL of 2 M sulfuric acid (Thermo Fisher Scientific, Boston, MA), and the absorbance was measured using a Tecan Infinite M200 Pro Nanoquant microplate reader (Männedorf, Switzerland) at 450 nm.

### Plant Material and Propagation In Vitro

Seeds of tobacco (Nicotiana tabactum) cultivar Wisconsin (obtained from the Victory Seed Company, Molalla, OR) were surface sterilized by immersion in 70% ethanol for 1 min, followed by soaking in 1.5% sodium hypochlorite for 10 min. After rinsing three times in sterile distilled water, and blotting dry with sterile filter paper, seeds were placed on germination medium MSG containing MS macro- and microelements with 1% sucrose and 0.7% agar (see Table 4 for details on media compositions). In vitro cultures were maintained by transferring 1-cm-long stem segments with axillary buds at 5-6 weeks intervals onto fresh MSP propagation medium.

TABLE 4
Media for tissue culture and transformation experiments.

| Media | Composition |
|---|---|
| MSG | Basic MS basal medium with 1% sucrose, 0.7% agar |
| MSP | MS with 3% sucrose, 0.7% agar |
| MSA | MS with 3% sucrose, 0.7% agar, 100 μM acetosyringone |
| MSRT | MS with 3% sucrose, 0.7% agar, 2 mg/L BAP, |
| | 0.1 mg/L NAA, 300 mg/L timentin, 100 mg/L |
| | kanamycin |
| MST | MS with 3% sucrose, 0.7% agar, 300 mg/L |
| | timentin, 100 mg/L kanamycin |
| MSCIT | MS with 3% sucrose, 0.7% agar, 1 mg/L 2,4 D, |
| | 0.5 mg/L NAA, 0.5 mg/L BAP, 300 mg/L |
| | timentin, 100 mg/L kanamycin |
| MSCT | MS with 3% sucrose, 0.7% agar, 2 mg/L 2.4-D, |
| | 200 mg/L timentin, 100 mg/L kanamycin |
| MSST | MS with 3% sucrose 2 mg/L 2.4-D 100 mg/L |
| | timentin, 100 mg/L kanamycin |

### Generation of Transgenic Plants

For plant transformation, DNA sequences encoding VHH specific for SARS-CoV-2 (GenBank, MT943599, VH ab8) were codon-optimized for plant expression. A synthetic cDNA encoding the VHH protein was cloned into the modified pBI121 binary vector (Jefferson et al. Embo J 1987, 6, 3901-3907) (Table 5 and FIG. 3) under a strong CaMV-35S promoter. The transformation vector included the nptII gene for kanamycin selection of the transgenic plants and HDEL. For purification of recombinant protein 6×Histidine and C-Myc tags were added. Vector pBI121-VHH was introduced into Agrobacterium tumefaciens strain LBA4404 for plant transformation experiments. Tobacco cv Wisconsin was transformed by Agrobacterium-mediated method according to Horsch (Horsch et al. Proc Natl Acad Sci USA 1986, 83, 2571-2575) with some modifications. Agrobacterium tumefaciens strain LBA4404 was grown on solid LB media supplemented with 50 mg/L kanamycin and 20 mg/L rifampicin. For transformation experiments, the agrobacterial cell suspension was prepared by inoculating 20 mL of the liquid LB medium with a single agrobacterial colony and grown for 2 days at 150 rpm on a rotary shaker at 25° C. The suspension of Agrobacterium was diluted with a liquid MS medium to obtain optical density of the solution at 600 nm (OD₆₀₀) close to 0.5 units. Leaf segments excised from 1-1.5-month-old tobacco plants were inoculated with Agrobacterium suspension with OD₆₀₀=0.5 for 10 min. After blotting dry with sterile filter paper, leaf segments were transferred to MSTC co-cultivation medium and incubated in the dark for 2 days at 23° C. Then tobacco explants were transferred to regeneration selection medium (MSRT, Table 4) containing 100 mg/L kanamycin. After 4-5 weeks of cultivation on MSRT medium, kanamycin-resistant green shoots (putative transformants) were formed. Healthy green shoots (1-2 cm) were excised and transferred to Phytatrays containing MST selection medium (Table 4) with 100 mg/L kanamycin.

TABLE 5
The amino acid sequences of SARS-COV-2 VHH
expressed by plants transformed with plasmid
pBI121 (shown in FIG. 3) used for plant
transfection. The bolded letters show the
position of the added c-Myc, and the under-
lined letters show the position of the 12x
His-tag. The lowercase letters show the
positions of the linkers.

| Vector Name | Vector Sequence |
|---|---|
| p81121-VHH- | EVQLVESGGGLVQPGGSLRLSCAASGFTFDDYAM |
| c-Myc-12xHis- | SWVRQAPGKGLEWIGRMYNNGRTSYNPSLKSLVT |
| tag | ISRDNSKNTLYLQMNSLRAEDTATYYCARDNLGY |
| | RPSENLYGMDVWGQGTTVTVSSaaaEQKLISEED |
| | LnsavdHHHHHHHHHHHH (SEQ ID NO: 4) |

### Callus Induction and Cell Suspensions

For callus induction, leaf segments were placed in 100×15 mm Petri dishes containing MSCI callus induction medium. Plates were incubated in the dark at 23° C. Well-developed tobacco callus tissues were selected and transferred to the callus propagation MSCT medium (Table 4) and incubated in the dark. For initiation of cell suspension, approximately 1 g of friable callus tissue was transferred into the sterile 250 mL conical flasks containing 50 mL of liquid MSST medium. Cell cultures were grown in the dark at 23° C., on a rotary shaker at 110 rpm. Maintenance of the cell suspension was carried out on the MSST medium with 10-day intervals for subcultures.

Protein Extraction from Plants and Plant Cell Suspensions

Proteins were extracted from transgenic tobacco leaf and plant cell suspensions that were engineered to express VHH nanobodies of interest. Respective samples (frozen tobacco leaves and cells) were ground to a fine dry powder in the presence of liquid nitrogen. Samples were resuspended in phosphate buffered saline (PBS) from Sigma Aldrich (Saint Louis, MO) and centrifuged at 20,000×g for 30 min. The supernatant was then vacuum filtered through a poly(ethersulfone) 0.2 μm pore size filtration cup (VWR, Radnor, PA). Samples were then frozen at −20° C. until further analysis.

### DNA Isolation and Real-Time Polymerase Chain Reaction (RT-PCR)

For extraction of the Tobacco cultivar Wisconsin plant DNA, DNeasy® Plant Mini Kit was used (Qiagen, Germantown, MD). Briefly, around 100 mg wet weight of plant tissues were disrupted using a mortar and pestle then the resulted materials were transferred to a microtube containing 400 μL of lysis buffer. Then, 4 μL RNase A was added to the solution, vortexed, and incubated for 10 min at 65° C. After the incubation, 130 μL of binding buffer was added to the mixture and incubated for 5 min on ice. In the next step, the lysate was centrifuged at 20,000×g for 5 min. Then, the lysate was pipet into a QIA shredder spin column placed in a 2 ml collection tube and centrifuged at 20,000×g for 2 min. In the next step, the flow-through solution was transferred into a new tube followed by the addition of 1.5 volumes of washing buffer. Then, 650 μL of the mixture was transferred into a DNeasy® Mini spin column placed in a 2 mL collection tube. The mixture was centrifuged at 6000×g for 1 min, the flow-through liquid was discarded and the procedure was repeated for the remaining sample. The spin column was then placed into a new 2 mL collection tube and washed two times with 500 μL of fresh washing buffer by centrifugation at 6000×g for 1 min and 20,000×g for 2 min. Then, the spin columns were transferred to a new 1.5 ml microcentrifuge tubes, 100 μL of elution buffer were added and the mixture was incubated at room temperature for 5 min. Finally, DNA was eluted at 6000×g for 1 min and the resulting DNA concentration was measured by Nanodrop 2000 spectrophotometer (Thermo Fisher Scientific, Boston, MA).

To perform PCR the following primers from IDT, Coralville, IA were used: 5′-GAAGTACAACTTGTTGAATCCGGTG-3′ (SEQ ID NO: 7) (forward) and 3′-TGAAGATACTGTCACGGITAGTACCT-5′(SEQ ID NO: 8) (reverse). The primers were designed to amplify the 372-base pair (bp) sequence of the target gene (VH ab8) excluding tags and stop codon. The PCR amplification of the VH ab8 gene was done using DreamTaq Green PCR Master Mix according to the manufacturer protocol (Thermo Fisher Scientific, Boston, MA). Cycling conditions were 95° C. for 1 min, followed by 35 cycles of 95° C. for 30 s, 55° C. for 30 s, 72° C. for 1 min and final extension at 72° C. for 7 min. Then, 10 μL of amplified DNA sample were loaded on 1% Agarose gel containing 0.4 μg/ml ethidium bromide (EtBr) and after electrophoresis bands were detected by ChemiDoc MP Imaging System (Bio-Rad, Hercules, CA).

### Statistical Analysis

Data were analyzed using descriptive statistics, single-factor analysis of variance (ANOVA), and presented as mean values±standard deviation (SD). The comparison among groups was performed by the independent sample student's t-test. The difference between variants was considered significant if P<0.05.

### Results

A VHH antibody that can bind to SARS-CoV-2 spike protein was produced in the WK6 bacterial system. For this purpose, the VHH genome sequence (GenBank Accession No. MT943599) with added cMYC and 12×His-tag elements was inserted into the pHEN6c vector using NcoI and EcoRI restriction sites. The resulting plasmid was transformed into the WK6 bacterial systems using a heat-shock method. The transfected bacteria were cultured in a culture plate with a selection marker (Carbenicillin 100 μg/mL), and selected colonies were checked for the expression of VHH-cMYC-12×His-tag using SDS-PAGE. Based on obtained results suitable colonies were selected for expansion. After expanding the selected colonies in LB Broth (containing 100 μg/mL Carbenicilin), the expression of the VHH was initiated by the addition of IPTG (0.4 mM). Then the protein was extracted and purified.

In the second step of this study, a single-chain VHH antibody that can bind to SARS-COV2 spike protein was produced in the EXPI293™ cells. For this purpose, the VHH genome sequence (GenBank Accession No. MT943599, VH ab8) with added cMYC and 12×His-tag elements were inserted into the pCDNA3.4™ vector using XbaI and AgeI restriction sites (Table 2 and FIG. 2). The produced proteins were extracted based on the affinity of 12 Histag to the Ni-NTA resin column. To evaluate the purity of extracted recombinant proteins, SDS-PAGE and PageBlue™ protein staining methods were used. The result showed high purity of the product. To confirm the SARS-CoV-2 VHH-cMYC-12×His-tag antibody expression in bacterial and mammalian cells, Western blot analysis was performed, and the results confirmed the expression of the desired product in bacterial and mammalian cells. Expression of the SARS-CoV-2 VHH in the WK6 bacterial cells was confirmed by SDS-PAGE. Expression of the SARS-CoV-2 VHH in the EXPI293F mammalian cells was confirmed by Western blot analysis.

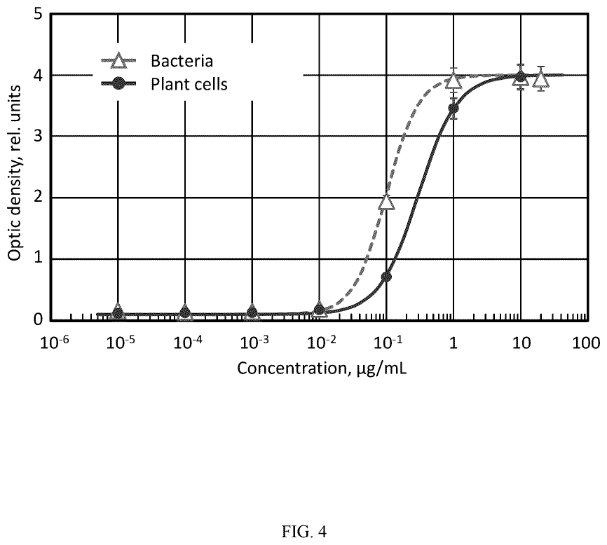

The ability of the VHH antibodies to bind the SARS-CoV-2 spike antigen was evaluated by ELISA. The results confirmed that the bacteria-derived VHH antibody was able to effectively bind the COVID-19 spike protein (FIG. 4). Similar results were obtained for VHH antibodies obtained in mammalian cells.

For plant transformation experiments seeds of tobacco (Nicotiana tabacum) cultivar Wisconsin were surface sterilized and placed on germination plant medium MSG (Table 4). Tobacco plants developed from the seeds were grown in Phytatrays™ (MilliporeSigma, Burlington, MA) under aseptic conditions. Leaves from one month old plants were used as explants for transformation experiments. For tobacco transformation, DNA sequences encoding VH ab8 specific for SARS-CoV-2 (GenBank, MT943599) were codon-optimized for plant expression. A synthetic cDNA encoding the VH ab8 protein was cloned into the modified pBI121 binary vector (FIG. 3) under CaMV-35 promoter, which is widely used as a strong constitutive promoter for transgene expression. The transformation vector also included the nptII gene for kanamycin selection of the transgenic plants and plant-specific endoplasmic reticulum retention signal (HDEL). For purification of recombinant protein 6×Histidine and C-Myc tags were added.

For stable transformation of tobacco, the Agrobacterium-mediated method was used, the most efficient for many plant species that use the agrobacteria as the biological vector to transfer exogenous T-DNA into the plant cell. The vector pBI121-VHH was introduced into Agrobacterium tumefaciens strain LBA4404 and used for tobacco transformation. Tobacco leaf segments were co-cultivated with Agrobacterium suspension on MSR medium and then transferred to regeneration selection MSRT medium (Table 4) containing 100 mg/L kanamycin and plant growth regulators for stimulation of shoot regeneration (2 mg/L 6-benzylaminopurine and 0.2 mg/L naphthaleneacetic acid). After 4-5 weeks of cultivation kanamycin-resistant shoots were developed and transferred for elongation and root development in a hormone-free MST medium (Table 4). Kanamycin-resistant tobacco plants demonstrated good growth and development on the selection medium, whereas non-transgenics were not able to grow, bleached, and died.

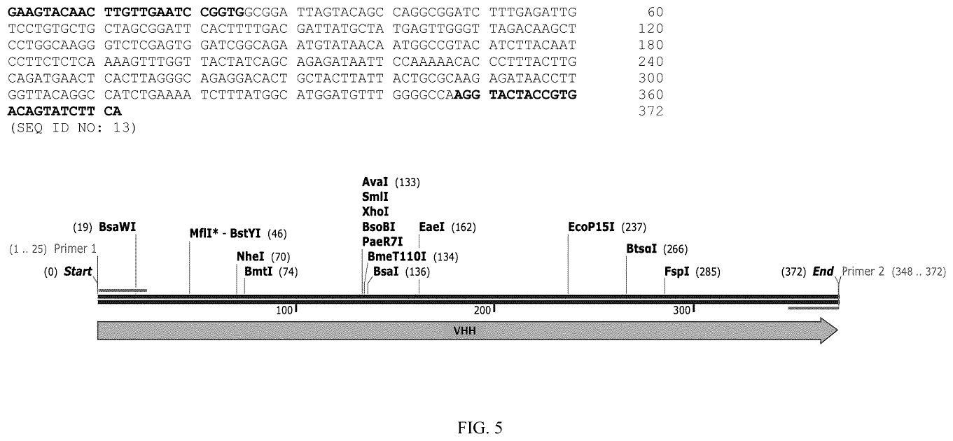

Eight independent tobacco kanamycin-resistant plants have been tested by PCR for the presence of the VH ab8 gene in genomic DNA. The incorporation of the VHH-antibody gene into plant genomes was confirmed by PCR analysis. The sequence of the PCR product is shown in FIG. 5. All eight transgenic plants showed the presence of the antibody's gene in plant DNA, whereas the control (non-transgenic plant) did not demonstrate the occurrence of this gene.

Another plant bioreactor is the cell suspension, which produces VHH antibody in sterile conditions. For the development of a cell suspension culture bioreactor, transgenic tobacco leaf explants were placed on callus induction media, in darkness. Various concentrations of plant growth regulators were tested for callus induction: 2,4-dichlorophenoxyacetic acid (2,4-D; 1-2 mg/L), naphthaleneacetic acid (NAA; 0.4-0.5 mg/L) and benzylaminopurine (BAP; 0.3-0.5 mg/L) Tobacco callus tissues (mass of undifferentiated cells) were initiated after 4-5 weeks of incubation. The most efficient medium for callus initiation was MSCI, contained 2 mg/L 2,4-D, 0.5 mg/L NAA and 0.5 mg/L BAP (Table 4). Then, primary callus tissues were transferred to and grown on callus propagation medium MSCT (Table 4). For initiation of cell suspension, fresh friable callus tissues were transferred into the sterile flasks containing liquid MSCS medium (Table 4). Cell suspensions were grown in the dark at 23° C., on a rotary shaker at 110 rpm. Maintenance of transgenic cell suspensions producing recombinant VHH antibodies was carried out on the MSCS medium with 10-day intervals for subcultures. Very fast-growing transgenic tobacco cell suspension was produced: about a 10-fold increase in cell volume was achieved in 9-10 days. Such efficient growth of cell suspension provides a very efficient system for the production of monoclonal antibodies, vaccines and other recombinant proteins.

Western blot analysis of several transgenic lines (plant and cell suspension) confirmed the presence of recombinant VHH antibodies with the expected molecular size at variable levels. The 15 kDa protein was detectable in several independent transgenic plant lines. The quantitative analysis of Western blots estimated the final concentration of VHH in the stock solution of 16.6 t 2.3 mg/mL.

A high level of expression of recombinant VHH protein was observed in cell suspensions. Several transgenic plants as well as cell suspensions were tested by ELISA. The binding of tobacco-derived VHH antibody to SARS-COV2 spike protein was confirmed by ELISA. Plan-derived antibodies demonstrated a similar binding activity to the COVID-19 spike protein when compared with antibodies derived from bacterial and mammalian cells.

In summary, the proof-of-concept experiments described herein for producing single chain VHH antibodies against the spike protein of the COVID-19 virus showed their viable production in plant systems of antibodies with a high ability to bind the targeted virus protein.

Nucleic Acid and Amino Acid Sequences
VHab8 amino acid sequence having GenBank
Reference No. MT943599
(SEQ ID NO: 1)
EVQLVESGGGLVQPGGSLRLSCAASGFTFDDYAMSWVRQAPGKGLEWIGR
MYNNGRTSYNPSLKSLVTISRDNSKNTLYLQMNSLRAEDTATYYCARDNL
GYRPSENLYGMDVWGQGTTVTVSS
Codon-optimized VHab8 nucleotide sequence
for plant expression
(SEQ ID NO: 2)
GAAGTACAACTTGTTGAATCCGGTGGCGGATTAGTACAGCCAGGCGGATC
TTTGAGATTGTCCTGTGCTGCTAGCGGATTCACTTTTGACGATTATGCTA
TGAGTTGGGTTAGACAAGCTCCTGGCAAGGGTCTCGAGTGGATCGGCAGA
ATGTATAACAATGGCCGTACATCTTACAATCCTTCTCTCAAAAGTTTGGT
TACTATCAGCAGAGATAATTCCAAAAACACCCTTTACTTGCAGATGAACT
CACTTAGGGCAGAGGACACTGCTACTTATTACTGCGCAAGAGATAACCTT
GGTTACAGGCCATCTGAAAATCTTTATGGCATGGATGTTTGGGGCCAAGG
TACTACCGTGACAGTATCTTCA
This codon-optimized nucleotide sequence
(SEQ ID NO: 2) encodes the same amino acid
sequence (SEQ ID NO: 1), but is different
from the original MT943599 sequence (different
nucleotide triplets can code the same amino
acid sequence).
Recombinant human coronavirus SARS-COV-2
Spike Glycoprotein RBD sequence. isolate
Wuhan-Hu-1, commercially available from
Abcam (Boston, MA)
(SEQ ID NO: 3)
RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGENCYFPLQSYGFQPTNGVGYQPYRVVVLSFELL
HAPATVCGPKKSTNLVKNKCVNFGSGGGSGGHHHHHH
VHab8 amino acid sequence (GenBank Reference
No. MT943599) with C-terminal tags c-Myc
and His-tag added (shown in Table 5)
(SEQ ID NO: 4)
EVQLVESGGGLVQPGGSLRLSCAASGFTEDDYAMSWVRQAPGKGLEWIGR
MYNNGRTSYNPSLKSLVTISRDNSKNTLYLQMNSLRAEDTATYYCARDNL
GYRPSENLYGMDVWGQGTTVTVSSaaaBQKLISEEDLnsavdHHHHHHHH
HHHH
Amino acid sequence of the SARS-COV-2 VHH
expressed by E. coli cells from the pHENC6-VHH
plasmid shown in FIG. 1
(SEQ ID NO: 5)
EVQLVESGGGLVQPGGSLRLSCAASGFTFDDYAMSWVRQAPGKGLEWIGR
MYNNGRTSYNPSLKSLVTISRDNSKNTLYLQMNSLRAEDTATYYCARDNL
GYRPSENLYGMDVWGQGTTVTVSSEQKLISEEDLHHHHHHHHHHHH
Amino acid sequence expressed by the SARS-
COV-2 VHH recombinant vectors pCDNA 3.4 VHH
of FIG. 2
(SEQ ID NO: 6)
EVQLVESGGGLVQPGGSLRLSCAASGFTFDDYAMSWVRQAPGKGLEWIGR
MYNNGRTSYNPSLKSLVTISRDNSKNTLYLQMNSLRAEDTATYYCARDNL
GYRPSENLYGMDVWGQGTTVTVSSaaaEQKLISEEDLasvadHHHHHHHH
HHHH
PCR primer (forward)
(SEQ ID NO: 7)
5′-GAAGTACAACTTGTTGAATCCGGTG-3′ (forward)
PCR primer (reverse)
(SEQ ID NO: 8)
3′-TGAAGATACTGTCACGGTAGTACCT-5′
Recombinant human coronavirus SARS-COV-2
Spike Glycoprotein RBD sequence of SEQ ID
NO: 3 without the His tag:
(SEQ ID NO: 9)
RVQPTESIVRFPNITNLCPFGEVENATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGENCYFPLQSYGFQPTNGVGYQPYRVVVLSFELL
HAPATVCGPKKSTNLVKNKCVNFGSGGGSGG
VHab8 amino acid sequence of SEQ ID NO: 4
without the His-tag:
(SEQ ID NO: 10)
EVQLVESGGGLVQPGGSLRLSCAASGFTFDDYAMSWVRQAPGKGLEWIGR
MYNNGRTSYNPSLKSLVTISRDNSKNTLYLQMNSLRAEDTATYYCARDNL
GYRPSENLYGMDVWGQGTTVTVSSaaaEQKLISEEDLasavd
Amino acid sequence of the SARS-COV-2 VHH
of SEQ ID NO: 5 without the His-tag:
(SEQ ID NO: 11)
EVQLVESGGGLVQPGGSLRLSCAASGFTEDDYAMSWVRQAPGKGLEWIGR
MYNNGRTSYNPSLKSLVTISRDNSKNTLYLQMNSLRAEDTATYYCARDNL
GYRPSENLYGMDVWGQGTTVTVSSEQKLISEEDL
Amino acid sequence of SEQ ID NO: 6 without
the His-tag:
(SEQ ID NO: 12)
EVQLVESGGGLVQPGGSLRLSCAASGFTFDDYAMSWVRQAPGKGLEWIGR
MYNNGRTSYNPSLKSLVTISRDNSKNTLYLQMNSLRAEDTATYYCARDNL
GYRPSENLYGMDVWGQGTTVTVSSaaaEQKLISEEDLasvad

### OTHER EMBODIMENTS

Any improvement may be made in part or all of the method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A transgenic plant or plant tissue or plant cell, or progeny thereof, comprising at least one polynucleotide integrated into the nuclear genome of the plant or plant tissue or plant cell, or progeny thereof, the at least one polynucleotide comprising at least one nucleotide sequence encoding a single variable domain of a heavy-chain antibody fragment (VHH) that specifically binds to a receptor binding domain of SARS-CoV-2 spike protein, wherein the VHH comprises amino acid sequence SEQ ID NO: 1 that is expressed in the transgenic plant or plant tissue or plant cell, or progeny thereof.

2. The transgenic plant or plant tissue or plant cell, or progeny thereof, of claim 1, wherein the at least one nucleotide sequence comprises SEQ ID NO: 2, the receptor binding domain of SARS-CoV-2 spike protein comprises the sequence of SEQ ID NO: 3, and the transgenic plant or plant tissue or plant cell, or progeny thereof, is selected from the group consisting of: tobacco, carrot, and cabbage plants.

3. The transgenic plant or plant tissue or plant cell, or progeny thereof, of claim 2, wherein the transgenic plant or plant tissue or plant cell, or progeny thereof, is selected from the group consisting of: Nicotiana tabactum, Daucus carota, and Brassica oleracea.

4. A tissue culture produced from protoplasts or cells or callus tissue from the transgenic plant or plant tissue or plant cell, or progeny thereof, of claim 1, wherein the protoplasts or cells or callus tissue are produced from a plant part selected from the group consisting of: leaves, pollen, embryos, cotyledon, hypocotyl, meristematic cells, roots, root tips, pistils, anthers, flowers, stems, glumes and panicles.

5. The tissue culture of claim 4, wherein the tissue culture comprises at least one bioreactor, and the transgenic plant or plant tissue or plant cell, or progeny thereof, is selected from the group consisting of: tobacco, carrot, and cabbage plants.

6. The tissue culture of claim 5, wherein the transgenic plant or plant tissue or plant cell, or progeny thereof, is selected from the group consisting of: Nicotiana tabacum, Daucus carota, and Brassica oleracea.

7. A method for producing transgenic tobacco, carrot, or cabbage plants comprising the steps of:
(a) stably transforming a plant or plant tissue or plant cell with at least one polynucleotide comprising at least one nucleotide sequence encoding a VHH that specifically binds to a receptor binding domain of SARS-CoV-2 spike protein under conditions such that the polynucleotide is integrated into the nuclear genome of the plant or plant tissue or plant cell;
(b) selecting stably transformed cells, shoots, callus cells, embryos or seeds;
(c) propagating transgenic plants from the stably transformed cells, shoots, callus cells, embryos or seeds selected in step (b); and
(d) selecting transgenic plants propagated in step (c) having the at least one polynucleotide integrated within the nuclear genome and expressing the VHH.

8. The method of claim 7, further comprising: (e) transferring the transgenic plants selected in step (d) to soil conditions allowing for self-pollination to generate homozygous transgenic plant lines, and wherein step (a) comprises Agrobacterium-mediated transformation.

9. The method of claim 7, wherein the transgenic tobacco, carrot, or cabbage plant is selected from the group consisting of: Nicotiana tabacum, Daucus carota, and Brassica oleracea, the at least one nucleotide sequence comprises SEQ ID NO: 2, and the receptor binding domain of SARS-CoV-2 spike protein comprises the sequence of SEQ ID NO: 3.

10. The method of claim 7, wherein transgenic plants are produced within 6-10 weeks.

11. The method of claim 10, wherein transgenic plants are produced within 8 weeks, and wherein at least step (c) is performed in a greenhouse for large-scale production of the transgenic tobacco, carrot, or cabbage plants, or in a field for large-scale production of the transgenic tobacco, carrot, or cabbage plants.

12. The method of claim 8, wherein the soil conditions are in a greenhouse or a field.

13. A method for producing VHH that specifically binds to a receptor binding domain of SARS-CoV-2 spike protein, the method comprising:
(a) providing the transgenic tobacco, carrot, or cabbage plants produced by the method of claim 8;
(b) propagating the transgenic tobacco, carrot, or cabbage plants in sterile conditions allowing continuous production of the VHH; and
(c) isolating the VHH from the transgenic tobacco, carrot, or cabbage plants.

14. The method of claim 13, wherein in step (c), the VHH is isolated from leaves of the transgenic tobacco, carrot, or cabbage plants, the step of stably transforming a plant or plant tissue or plant cell comprises Agrobacterium-mediated transformation, and the plant or plant tissue or plant cell, or the transgenic seeds, are a transgenic tobacco, carrot, or cabbage plant or seed.

15. The method of claim 14, wherein the transgenic tobacco, carrot, or cabbage plant or seed is selected from the group consisting of: Nicotiana tabacum, Daucus carota, and Brassica oleracea.

16. The method of claim 13, wherein the at least one nucleotide sequence comprises SEQ ID NO: 2, and the receptor binding domain of SARS-CoV-2 spike protein comprises the sequence of SEQ ID NO: 3.

17. A method for producing VHH that specifically binds to a receptor binding domain of SARS-CoV-2 spike protein, the method comprising:
(a) culturing transgenic plant tissue according to claim 1 in a first callus medium comprising at least one plant hormone for initiation of primary compact callus;
(b) culturing the primary callus in the dark for a period sufficient to result in a primary compact callus;
(c) transferring the primary compact callus to a second callus medium and propagating the primary compact callus until friable callus comprising loosely attached cells develops;
(d) culturing the friable callus comprising loosely attached cells in liquid maintenance medium as a cell suspension; and
(e) collecting cells from the cell suspension and isolating the VHH.

18. The method of claim 17, wherein step (d) is performed in at least one bioreactor, and the transgenic plant tissue is at least one leaf segment.

19. The method of claim 17, wherein the at least one nucleotide sequence comprises SEQ ID NO: 2, the receptor binding domain of SARS-CoV-2 spike protein comprises the sequence of SEQ ID NO: 3, and the transgenic plant tissue is selected from the group consisting of: tobacco, carrot, and cabbage plants.

20. The method of claim 19, wherein the transgenic plant tissue is selected from the group consisting of: Nicotiana tabactum, Daucus carota, and Brassica oleracea.
